(19) United States
(12) Patent Application Publication
(10) Pub. No.: US 2020/0323225 A1
(43) Pub. Date: Oct. 15, 2020

(54) LIPASE INHIBITORS TO PREVENT RANCIDITY IN EXPRESSED HUMAN MILK DURING STORAGE

(71) Applicants: Elani Fourie Wiest, Albuquerque, NM (US); Katherine E Zychowski, Albuquerque, NM (US)

(72) Inventors: Elani Fourie Wiest, Albuquerque, NM (US); Katherine E Zychowski, Albuquerque, NM (US)

(73) Assignee: STC.UNM, Albuquerque, NM (US)

(21) Appl. No.: 15/733,281

(22) PCT Filed: Dec. 19, 2018
(86) PCT No.: PCT/US2018/066477

§ 371 Date: Jun. 19, 2020

Related U.S. Application Data
(60) Provisional application No. 62/607,699, filed on Dec. 19, 2017.

(51) Int. Cl. A23C 3/00; C07K 16/40; A61M 1/06; A23C 9/20; A23L 3/3499; A23L 33/10; A23L 33/00

(52) U.S. Cl. CPC A23C 3/00; C07K 16/40; A61M 1/062; A23V 2002/00; A23L 3/3499; A23L 33/10; A23L 33/40; A23C 9/206

(57) ABSTRACT

Methods and apparatus for improving expressed human milk (EHM0 shelf-life as well as maintaining or improving EHM taste and smell comprising the utilization of specific lipase inhibitors and antibodies.

### CROSS-REFERENCE TO RELATED APPLICATIONS

The following application claims benefit of U.S. Provisional Application No. 62/607,699, filed Dec. 19, 2017, which is hereby incorporated by reference in its entirety.

### BACKGROUND

It is well-known that breastfeeding is unequivocally the ideal source for infant nutrition [1-5]. The American Academy of Pediatrics (AAP) recommends breast feeding exclusively for 6 months of life followed by introduction of solid foods with continued breast feeding for at least one year [6]. The 2010 United States Census determined that nearly 70% of mothers work outside of the home [7], resulting in breast feeding mothers needing to routinely express milk during the day, during separation from their infants [8]. Expressed human milk (EHM) is then subsequently packaged in a bottle or bag and refrigerated or frozen for further use [9].

The American Academy of Pediatrics has stated that the supply of infant nutrition is “ . . . a public health issue and not only a lifestyle choice . . . [g]iven the documented short- and long-term medical and neurodevelopmental advantages of breastfeeding.” (AAP) Human milk contains a number of components that are anti-microbial [34]. Heat treatment (i.e. scalding, pasteurization used to prevent lipolysis) can damage membranes surrounding milk fat globules [35]. This fragmentation allows lipases to further degrade triglycerides, thus causing additional alterations in EHM. Therefore, subjecting EHM to heat treatment is detrimental to preserving many of its beneficial, nutritional attributes. While “typical” lipase activity is currently unknown, there is substantial variation in milk triglyceride content, which composes 98% of total lipids in human milk [25]. After only two freeze-thaw cycles, levels of linoleic acid is significantly decreased in addition to (non-significant) decreases in oleate, palmitate, stearate, and myristate after only one freeze-thaw cycle [25].

Moreover, it is well known that early flavor and odor learning can subsequently influence feeding behavior [36](Beauchamp et al. 2009). Additionally, breast milk is a direct reflection of the odors and flavors in a mother's diet [37]. Sweetness and textural properties of breast milk including viscosity and mouth coating vary widely [38]. Infants fed breast milk, milk-based formula, or hydrolyzed casein formula all have differing tolerances and preferences for sweet, bitter, sour, salty and umami as they mature during childhood ([39, 40]. It has been documented that the milk falvor with an elevated FFA level greater than 1.5 mmol L−1 (which include lauric and capric acid), is considered unacceptable by most individuals [41], and may result in infant refusal.

Proper storage of EHM is critical for infant nutrition. It is currently recommended that breast milk must be consumed within 5 hours at room temperature, 5 days under refrigeration (4° C.) or within 5 months of freezing (−20° C.) [10]. Depending on the type and length of storage, EHM may lose some of its functional and nutritional properties [11-14]. Like all mammalian milk, EHM contains lipases that gradually breakdown fatty acids over time. There are two primary EHM lipases: bile salt-dependent lipase (BSDL) and serum-stimulated lipase, also known as lipoprotein lipase (LPL) [15-16]. BSDL has been documented to improve infant lipid digestion and absorption, playing a key role in the breakdown of retinol esters, thereby providing bioavailable vitamin A to the infant [17]. However, unlike BSDL which plays a critical role in the infant gut, LPL has no known function in neonatal digestion. More exactly, it is involved in uptake of circulating fatty acids into the maternal mammary gland [18, 19]. Following consumption, LPL likely degrades upon ingestion due to low pH in the stomach [20].

Excess lipase activity has been implicated in rancidity in mammalian milk [21-23], causing molecular alterations such as an increase in free fatty acids and subsequent rancid off-flavors and odors [24] that are unacceptable to most infants. Lipolysis and subsequent change in odor and flavor profile is a constant issue in the dairy industry, though it is mostly mitigated through the commercial pasteurization process [21, 25], which destroys most lipases. Analogously, mothers with perceived excess lipase in their EHM are encouraged to scald EHM [26], however this is challenging, time-consuming, results in molecular and biochemical, nutritionally-relevant alterations [25, 27, 28] and loss of EHM volume, which are all critical for the infant.

Moreover, mothers face numerous obstacles breast feeding which may lead to early cessation. Insufficient maternity leave [29], overfeeding EHM by caretakers [8, 30, 31], perceived EHM insufficiency [32], and limited public and familial support are all contributing factors to early breast feeding cessation. Travel, challenging pumping conditions and timing can also lead to less than ideal EHM storage situations. Freezing EHM is a common practice for working mothers, since it is considered to be safe and slow the growth of bacteria [33]. However, EHM may develop a rancid flavor after frozen storage as a result of lipolysis and a significant increase in total free fatty acids (FFA), capric acid, and lauric acid [23].

Accordingly, there is a need for a breast milk storage solution that is more robust to environmental stresses, including freezer storage, while retaining desirable flavor and nutritional benefits.

### SUMMARY

The present disclosure provides methods and apparatus for improving EHM shelf-life as well as maintaining or improving EHM taste and smell comprising the utilization of specific lipase inhibitors and antibodies. In particular, the present disclosure provides methods and apparatus for lipase-reducing interventions.

### BRIEF DESCRIPTION OF THE DRAWINGS

FIG. 1 is a schematic illustration of an exemplary lipase inhibiting EHM storage bag.

FIG. 1a is a close up of the side of the EHM storage bag of FIG. 1.

FIG. 2 shows lipolysis of triacylglycerol into glycerol and then free fatty acids including lauric acid and capric acid.

FIG. 3 is a graph showing measurement of LPL activity in milk spiked with 6 pmol LPL in the absence or presence of LPL inhibitor, Orlistat.

FIG. 4 is a graph sowing lipase activity inhibition after incubation with Orlistat. Raw milk samples (n=2) were incubated with 10 μM Orlistat for 1 h which resulted in a significant (22%) decrease in LPL.

### DETAILED DESCRIPTION

According to an embodiment the present disclosure provides methods and apparatus for improving EHM shelf-life as well as maintaining or improving EHM taste and smell comprising the utilization of specific lipase inhibitors. In particular, the present disclosure provides methods and apparatus for lipase-reducing interventions. According to a specific embodiment, the present disclosure provides for the addition of lipase-inhibitors and/or antibodies directly to EHM. According to another specific embodiment, the present disclosure provides an EHM storage container that reduces or eliminates some or all of the negative consequences of the presence of lipase in EHM. According to a still further embodiment, the present disclosure provides a device having lipase inhibitors and/or lipase-specific antibodies immobilized to at least a portion of a surface such that EHM that passes over or otherwise contacts the surface is exposed to the lipase inhibitors and/or lipase-specific antibodies.

For the purposes of the present disclosure, the term “lipase inhibitor” is intended to mean a molecule which is able to specifically bind to a lipase of interest (LPL for example), thereby preventing lipolysis.

As discussed above, EHM contains many different lipases, some of which, like BSDL, are known to be beneficial to neonatal health while others, like LPL, have no known benefit to the infant. Accordingly, unlike pasteurization, which destroys all lipases present in EHM, an embodiment of the present disclosure provides for utilization of specific lipase inhibitors and/or antibodies, which can reduce or eliminate the presence or activity of only certain undesirable lipases, or “lipases of interest”. For example, various embodiments may use lipase inhibitors or antibodies that do not have any known positive effect on infant health or only those lipases that are known to have a negative effect on EHM. As a specific example, an embodiment of the present disclosure might utilize lipase inhibitors or antibodies that have been found to be effective against LPS but ineffective against BSDL. Alternatively, or additionally, an embodiment of the present disclosure might utilize lipase inhibitors or antibodies that have been found to be prevent or inhibit fatty acid synthesis, lipolysis, or other activities and/or molecules that are associated with unpleasant flavors, rancidity, or other identified factors. Examples of molecules which may be targeted due to their associate with unpleasant flavors include, but are not limited to, capric acid and lauric acid (See, e.g., FIG. 2). Table 1 provides a list of fatty acids and their formulas.

TABLE 1

| Fatty AcidsFlavor Analysis Panel | Formula |
|---|---|
| Capric | C10:0 |
| Lauric | C12:0 |
| Myristic | C14:0 |
| Palmitic | C16:0 |
| Palmitelaidic | C16:1n7t |
| Palmitoleic | C16:1n7 |
| Stearic | C18:0 |
| Elaidic | C18:1t |
| Oleic | C18:1n9 |
| Linoelaidic | C1:2n6t |
| Linoleic | C18:2n6 |
| Arachidic | C20:0 |
| Gamma-Linolenic | C18:3n3 |
| Eicosenoic | C20:1n9 |
| Alpha-Linolenic | C18:3n3 |
| Eicosadienoic | C20:2n6 |
| Behenic | C22:0 |
| Dihomo-g-linolenic | C20:3n6 |
| Arachidonic | C20:4n6 |
| Ligneceric | C24:0 |
| Eicosapentaenoic | C20:5n3 |
| Nervonic | C24:1n9 |
| Docosapentaenoic | C22:4n6 |
| Docosapentaenoic - n6 | C22:5n6 |
| Docosapentaenoic - n3 | C22:5n3 |
| Docosahexaenoic | C22:6n3 |

Suitable lipase inhibitors or lipase specific antibodies include both naturally occurring and synthetic lipases and antibodies. Examples of lipase inhibitors include, but are not limited to, GSK264220A, Orlistat, RHC 80267, and Xen 445. An excellent discussion of lipases and lipase inhibitors is provided in Bialecka-Florjanczyk et al., “Synthetic and Natural Lipase Inhibitors” Mini-Reviews in Medicinal Chemistry, 2018, Vol. 18, No. 8. Examples of suitable antibodies include, but are not limited to, ab137821 (Abc am), MAb 5D2 or any antibody that will bind to and inhibit LPL from degrading lipids into fatty acids.

According to a first embodiment, the present disclosure provides for the addition of lipase inhibitors or lipase-specific antibodies directly to EHM. For example, an amount of lipase inhibitor may be added to a container containing EHM under sufficient conditions that the EHM is able to interact with and thus bind to the lipase inhibitors. For the purposes of the present disclosure, and unless specifically stated to the contrary, the term “container” is intended to include any item that is able to contain EHM either temporarily or for storage, including, but not limited to, bottles, bags, plasticware, glassware, etc.

According to a further embodiment, rather than adding the lipase inhibitors directly to the EHM, the present disclosure provides a container having lipase inhibitors or antibodies immobilized to at least a surface of the container (referred to herein as an anti-lipase coating). In this embodiment, because the lipase inhibitor is immobilized to the surface of the container, any lipase in the EHM that is exposed to the lipase inhibitor will bind to the immobilized lipase inhibitor and thus bound to the container surface as well. The resulting lipase-free EHM can then be fed directly to an infant or transferred to another storage container. It will be understood, or course, that the term “lipase-free” in this context is intended to indicate that at least some of the lipase of interest in the EHM has been bound or altered in such a way that the negative effects of the presence of the lipase of interest on EHM are reduced or eliminated. As stated above, the anti-lipase coating may contain lipase-inhibitors or antibodies that specifically or preferentially inhibit only some lipases.

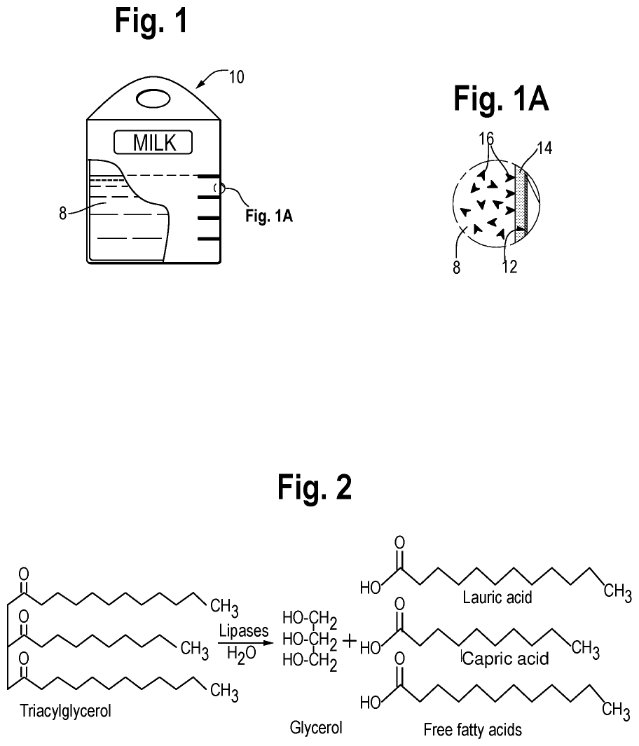

An exemplary lipase-inhibiting EHM storage bag 10 is shown in FIG. 1. As shown in close-up FIG. 1A, at least some of the inner surface 12 of the bag is coated with a lipase inhibitor or antibody 14. The anti-lipase coating binds active lipases 16 in milk 8 stored in the bag, thereby reducing molecular alterations caused by and/or other negative consequences of the presence of lipase in EHM. According to a specific embodiment, the present disclosure contemplates the production, distribution, and use of disposable lipase inhibitor-coated EHM storage bags in order to increase EHM shelf life and/or improve taste, thereby encouraging the use of EHM in infant care.

According to a still further embodiment, the lipase-inhibiting coating is applied to at least a portion of a device surface to which the EHM will be directly exposed some time prior to use (i.e. feeding of the EHM to an infant.) Suitable devices include, but are not limited to, any surface of a breast pump which makes direct contact with the EHM, including tubing, suction cups, bags, and/or containers, as well as filters, funnels, tubing, bottles, nipples, or other apparatus that might be used for collection, storage, or delivery of EHM (whether delivery is directly to an infant or to another container). According to this embodiment, the device may be made of any suitable material including, but not limited to plastics and/or glass.

It will be appreciated that the specific mechanism by which the lipase inhibitor is immobilized to the surface will be determined by multiple factors including, but not limited to, the specific lipase inhibitor being immobilized, the material that forms the surface, expected temperature range, and characteristics of that material including, but not limited to, whether the material is rigid (like a bottle) or flexible (like a storage bag). For example, coatings used for storage bags would likely be expected to maintain their ability to immobilize lipase inhibitors in temperatures that are below freezing (for storage) and as high as body temperature (for feeding). Of course, some surfaces may be intended for use at more limited temperature ranges (i.e. surfaces for use with a breast pump may not need to include coatings that are sufficient at freezing temperatures.) It should be further noted that the lipase-inhibiting coating may not successfully immobilize all lipase inhibitors at all times, there my be some release or separation under some conditions, but the general intent would be for the coating to generally retain the lipase of interest during collection, storage and/or delivery of the EHM (again, whether delivery is directly to an infant or to another container).

Suitable polymer coatings may include polyethylene glycol or cholesterol-based or -derived coatings such as, though not limited to, those described in, for example, Tiller et al., Designing surfaces that kill bacteria on contact” PNAS, May 22, 2001 Vol. 98, no. 11, pp 5981-5985; Kim et al., “From Self-Assembled Monolayers to Coatings: Advances in the Synthesis and Nanobio Applications of Polymer Brushes,” Polymers 2015, 7, 1346-1378; and Sidenbiedel et al., “Antimicrobial Polymers in Solution and on Surfaces: Overview and Functional Principles” Polymers 2012, 4, 46-71; as well as U.S. Provisional Patent Application No. 62/779,665, filed Dec. 14, 2018. Of course it will be appreciated that some of the above-described coatings may be better suited for certain applications over others and may, thus, require certain modifications. For example, a coating which is typically used to retain only a certain molecule, only on certain types of surfaces (i.e. rigid), only on certain materials, or only for the short term (i.e. for the purposes of drug delivery) might need to be modified for the present purposes of retention of certain lipases of interest. Accordingly, it will be appreciated that the above-identified list is provided only as general guidance with regards to various types of coatings that could be utilized or modified for the presently described purposes.

It will be appreciated that while the present disclosure has been directed primarily towards expressed human breast milk and the collection and storage thereof, the presently described methods and devices could similarly be applied to other substances including food and food products (including, but not limited to non-human derived milk), particularly, though not necessarily, when such foods or food products are not pasteurized.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

All patents and publications referenced below and/or mentioned herein are indicative of the levels of skill of those skilled in the art to which the invention pertains, and each such referenced patent or publication is hereby incorporated by reference to the same extent as if it had been incorporated by reference in its entirety individually or set forth herein in its entirety. Applicants reserve the right to physically incorporate into this specification any and all materials and information from any such cited patents or publications.

### REFERENCES

1. Leon-Cava, N., et al., Quantifying the benefits of breastfeeding: a summary of the evidence. Pan American Health Organization, Washington D.C., 2002.
2. Heinig, M. J., Host defense benefits of breastfeeding for the infant: effect of breastfeeding duration and exclusivity. Pediatric Clinics of North America, 2001. 48(1): p. 105-123.
3. Victora, C. G., et al., Breastfeeding in the 21st century: epidemiology, mechanisms, and lifelong effect. The Lancet, 2016. 387(10017): p. 475-490.
4. Binns, C., M. Lee, and W. Y. Low, The long-term public health benefits of breastfeeding. Asia Pacific Journal of Public Health, 2016. 28(1): p. 7-14.
5. Tudehope, D. I., Human milk and the nutritional needs of preterm infants. The Journal of pediatrics, 2013. 162(3): p. S17-S25.
6. Gartner, L. M., et al., Breastfeeding and the use of human milk. Pediatrics, 2005. 115(2): p. 496-506.
7. Murtagh, L. and A. D. J. A. J. o. P. H. Moulton, Working mothers, breastfeeding, and the law. 2011. 101(2): p. 217-223.
8. Rasmussen, K. M. and S. R. Geraghty, The quiet revolution: breastfeeding transformed with the use of breast pumps. American journal of public health, 2011. 101(8): p. 1356-1359.
9. Cheng, D., E. O'Sullivan, and K. Rasmussen, Maternal beliefs and practices about storage of human milk. The FASEB Journal, 2015. 29(1 Supplement): p. 583-2.
10. Wilson-Clay, B. and K. Hoover, The breastfeeding atlas. 2008: LactNews Press Texas.
11. Williamson, M. T. and P. K. Murti, Effects of storage, time, temperature, and composition of containers on biologic components of human milk. Journal of Human Lactation, 1996. 12(1): p. 31-35.
12. Lawrence, R. A., Storage of human milk and the influence of procedures on immunological components of human milk. Acta Paediatrica, 1999. 88(s430): p. 14-18.
13. Tully, D. B., F. Jones, and M. R. Tully, Donor milk: what's in it and what's not. Journal of Human Lactation, 2001. 17(2): p. 152-155.
14. Heiman, H. and R. J. Schanler, Benefits of maternal and donor human milk for premature infants. Early human development, 2006. 82(12): p. 781-787.
15. BlÄCkberg, L. and O. Hernell, The Bile-Salt-Stimulated Lipase in Human Milk. The FEBS Journal, 1981. 116(2): p. 221-225.
16. Zechner, R., Rapid and simple isolation procedure for lipoprotein lipase from human milk. Biochimica et Biophysica Acta (BBA)-Lipids and Lipid Metabolism, 1990. 1044(1): p. 20-25.
17. Manson, W. G. and L. T. Weaver, Fat digestion in the neonate. Archives of Disease in Childhood-Fetal and Neonatal Edition, 1997. 76(3): p. F206-F211.
18. Hamosh, M. and P. Hamosh, Development of digestive enzyme secretion. Development of the Gastrointestinal Tract, 2000: p. 261-277.
19. Clark, R. M., et al., Effect of temperature and length of storage on serum-stimulated and serum-independent lipolytic activities in human milk. Journal of pediatric gastroenterology and nutrition, 1984. 3(4): p. 567-570.
20. Liao, T. H., P. Hamosh, and M. Hamosh, Fat digestion by lingual lipase: mechanism of lipolysis in the stomach and upper small intestine. Pediatric research, 1984. 18(5): p. 402-409.
21. Deeth, H. C., Lipoprotein lipase and lipolysis in milk. International Dairy Journal, 2006. 16(6): p. 555-562.
22. Castberg, H. B., et al., Lipases in bovine milk and the relationship between the lipoprotein lipase and tributyrate hydrolysing activities in cream and skim-milk. Journal of Dairy Research, 1975. 42(2): p. 255-266.
23. Hung, H.-Y., et al., Variations in the rancid-flavor compounds of human breastmilk under general frozen-storage conditions. BMC pediatrics, 2018. 18(1): p. 94.
24. Sørensen, A.-D. M., et al., Human milk fat substitute from butterfat: production by enzymatic interesterification and evaluation of oxidative stability. Journal of the American Oil Chemists' Society, 2010. 87(2): p. 185-194.
25. Henderson, T. R., T. N. Fay, and M. Hamosh, Effect of pasteurization on long chain polyunsaturated fatty acid levels and enzyme activities of human milk. The Journal of pediatrics, 1998. 132(5): p. 876-878.
26. Lawrence, R. A. and R. M. Lawrence, Biochemistry of human milk. Breastfeeding. A guide for the medical profession. 6th ed. Philadelphia: Elsevier Mosby, 2005: p. 105-70.
27. Wardell, J. M., et al., Bile salt-stimulated lipase and esterase activity in human milk after collection, storage, and heating: nutritional implications. Pediatric Research, 1984. 18(4): p. 382-386.
28. Muir, D. D., The shelf-life of dairy products: 1. Factors influencing raw milk and fresh products. International Journal of Dairy Technology, 1996. 49(1): p. 24-32.
29. Baker, M. and K. Milligan, Maternal employment, breastfeeding, and health: Evidence from maternity leave mandates. Journal of health economics, 2008. 27(4): p. 871-887.
30. Dewey, K. G., et al., Maternal versus infant factors related to breast milk intake and residual milk volume: the DARLING study. Pediatrics, 1991. 87(6): p. 829-837.
31. Li, R., S. B. Fein, and L. M. Grummer-Strawn, Do infants fed from bottles lack self-regulation of milk intake compared with directly breastfed infants? Pediatrics, 2010. 125(6): p. e1386-e1393.
32. Winikoff, B., et al., Overcoming obstacles to breast-feeding in a large municipal hospital: applications of lessons learned. Pediatrics, 1987. 80(3): p. 423-433.
33. Schanler, R. J., N. F. Krebs, and S. B. Mass, Breastfeeding handbook for physicians. 2013: American Academy of Pediatrics.
34. May, J. T., Antimicrobial properties and microbial contaminants of breast milk-an update. Journal of Paediatrics and Child Health, 1984. 20(4): p. 265-269.
35. Wardell, J. M., C. M. Hill, and S. W. Souza, Effect of pasteurization and of freezing and thawing human milk on its triglyceride content. Acta Pædiatrica, 1981. 70(4): p. 467-471.
36. Beauchamp, G. K. and J. A. Mennella, Early flavor learning and its impact on later feeding behavior. Journal of pediatric gastroenterology and nutrition, 2009. 48: p. S25-S30.
37. Galef, B. G. and D. F. Sherry, Mother's milk: a medium for transmission of cues reflecting the flavor of mother's diet. Journal of comparative and physiological psychology, 1973. 83(3): p. 374.
38. Barker, E., Sensory evaluation of human milk. Unpublished masters thesis, University of Manitoba, Winnipeg, Canada, 1980.
39. Mennella, J. A., et al., Early milk feeding influences taste acceptance and liking during infancy—. The American journal of clinical nutrition, 2009. 90(3): p. 780S-788S.
40. Mennella, J. A. and G. K. Beauchamp, Flavor experiences during formula feeding are related to preferences during childhood. Early human development, 2002. 68(2): p. 71-82.
41. Downey, W. K. J. J. o. D. R., Flavour impairment from pre-and post-manufacture lipolysis in milk and dairy products. 1980. 47(2): p. 237-252.

### EXPERIMENTAL

### LPL Activity and Orlistat Inhibition

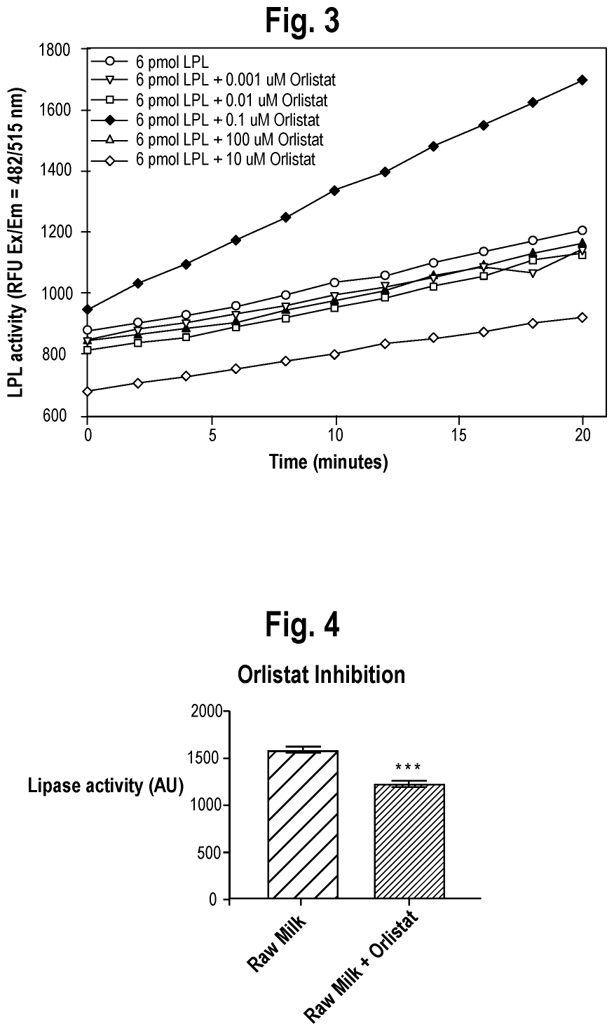

LPL activity in milk spiked with 6 pmol LPL and increasing levels of Orlistat (an LPL inhibitor) was monitored over the course of 20 min (FIG. 3). As expected, 10 μM Orlistat resulted in the most significant reduction in LPL. Raw milk samples were also treated with this level of Orlistat (10 μM) (FIG. 4). This in vitro inhibition resulted in a significant 22% decrease in lipase activity.

Coating of Commercial Milk Storage Bags with Cholesterol

Sections (2 cm2) of commercial breast milk storage bags (Medela LLC, McHenry, IL) were cut to size (n=3) for coating with cholesterol (a milk/storage bag compatible coating vehicle). Cholesterol (5% w/v) was dissolved into heated ethanol (50° C.). The pre-weighed bag sections were taped to a backing card and coated in a fume hood. Spray coating was performed using a Master Airbrush® System model G22 airbrush apparatus and a compressor (TCP Global, San Diego, Calif.). Each single sweep coating application was followed by 2 minutes solvent evaporation time, this was repeated in triplicate. Cholesterol weight gain over time with bag sections was determined (Table 2). Visual comparison of section of the storage bags showed minimal impact from the cholesterol coating.

TABLE 2
Preliminary coating data. Polyethylene breast
milk storage bags with 3-layers of cholesterol

| | Start Weight | End Weight | Weight gain | |
|---|---|---|---|---|
| Sample | (mg) | (mg) | (mg) | % Weight gain |
| 1 | 138 | 140 | 2 | 1.4 |
| 2 | 137 | 141 | 4 | 2.9 |
| 3 | 126 | 136 | 10 | 7.9 |
| | | | Mean | 4.1 |
| | | | SD | 3.4 |

What is claimed is:

1. An expressed human milk (EHM) storage container comprising one or more lipase inhibitors or lipase-specific antibodies immobilized to the surface.

2. The storage container of claim 1 wherein the lipase inhibitor or lipase-specific antibody does not inhibit or interfere with bile salt-dependent lipase (BSDL).

3. The storage container of claim 1 wherein the lipase inhibitor or lipase-specific antibody is directed towards lipoprotein lipase (LPL).

4. The storage container of claim 1 wherein the one or more lipase inhibitors and/or one or more lipase-specific antibodies are selected from the group consisting of: GSK264220A, Orlistat, RHC 80267, Xen 445, ab137821 (Abcam), and MAb 5D2.

5. A device for improving shelf-life and/or maintaining and/or improving taste and/or smell in expressed human milk (EHM) comprising surface having one or more lipase inhibitors and/or one or more lipase-specific antibodies immobilized to the surface.

6. The device of claim 5 wherein the lipase inhibitor or lipase-specific antibody does not inhibit or interfere with bile salt-dependent lipase (BSDL).

7. The device of claim 5 wherein the lipase inhibitor or lipase-specific antibody is directed towards lipoprotein lipase (LPL).

8. The device of claim 5 wherein the one or more lipase inhibitors and/or one or more lipase-specific antibodies are selected from the group consisting of: GSK264220A, Orlistat, RHC 80267, Xen 445, ab137821 (Abcam), and MAb 5D2.

9. The device of claim 5 wherein the device is part of a mechanical breast pump.

10. The device of claim 5 wherein the device is a filter.

11. A method for improving shelf-life and/or maintaining and/or improving taste and/or smell in expressed human milk (EHM) comprising exposing EHM to one or more lipase inhibitors and/or one or more lipase-specific antibodies.

12. The method of claim 11 wherein the lipase inhibitor or lipase-specific antibody does not inhibit or interfere with bile salt-dependent lipase (BSDL).

13. The method of claim 11 wherein the lipase inhibitor or lipase-specific antibody is directed towards lipoprotein lipase (LPL).

14. The method of claim 11 wherein exposing comprises delivering an amount of lipase inhibitor or lipase-specific antibody to EHM within a container.

15. The method of claim 11 wherein exposing EHM comprises providing a container for EBM wherein the container contains one or more lipase inhibitors or lipase-specific antibodies.

16. The method of claim 11 wherein exposing EHM comprises providing a surface having one or more lipase inhibitors and/or one or more lipase-specific antibodies immobilized thereto and exposing the EHM to the surface.

17. The method of claim 16 wherein the surface is at least a portion of the inside of a container.

18. The method of claim 16 wherein the surface is at least a portion of a filter.

19. The method of claim 16 wherein the surface is at least a portion of a pump.

20. The method of claim 11 wherein the one or more lipase inhibitors and/or one or more lipase-specific antibodies are selected from the group consisting of: GSK264220A, Orlistat, RHC 80267, Xen 445, ab137821 (Abcam), and MAb 5D2.

21. A method for treating expressed breast milk comprising exposing the breast milk to a lipase inhibitor or antibody directed towards lipases or fatty acids that are known to affect taste, wherein the lipase inhibitor or antibody is not directed towards lipases or fatty acids that have known health benefits.

22. The method of claim 21 wherein the lipase inhibitor or antibody is immobilized to a storage device for, or a device used for expression of, the expressed breast milk.

23. A method for treating non-pasteurized food or food products comprising exposing the food or food products to a specific lipase inhibitor or antibody, wherein the lipase inhibitor or antibody is immobilized to a storage device for the food or food product.

24. A device for storing a food or food product comprising a lipase inhibitor or lipase-specific antibody immobilized to the surface of the device.
